(19) United States
(12) Patent Application Publication
(10) Pub. No.: US 2022/0105487 A1
(43) Pub. Date: Apr. 7, 2022

(54) SPRAY DRYING METHODS FOR ENCAPSULATION OF OXYGEN LABILE CARGO IN CROSS-LINKED POLYMER MICROCAPSULES

(71) Applicant: THE REGENTS OF THE UNIVERSITY OF CALIFORNIA, Oakland, CA (US)

(72) Inventors: Scott Strobel, Davis, CA (US); Herbert B. Scher, Moraga, CA (US); Tina Jeoh Zicari, Davis, CA (US)

(73) Assignee: THE REGENTS OF THE UNIVERSITY OF CALIFORNIA, Oakland, CA (US)

(21) Appl. No.: 17/501,745

(22) Filed: Oct. 14, 2021

Related U.S. Application Data
(63) Continuation of application No. PCT/US2020/029557, filed on Apr. 23, 2020.
(60) Provisional application No. 62/837,399, filed on Apr. 23, 2019.

(51) Int. Cl. B01J 13/14; C08L 5/04; C08L 3/06; C08K 5/09; B01J 13/04; C08K 3/28

(52) U.S. Cl. CPC B01J 13/14; C08L 5/04; C08K 3/28; C08K 5/09; B01J 13/043; C08L 3/06

(57) ABSTRACT

Systems and methods are provided for microencapsulating oxygen sensitive cargo such as polyunsaturated fatty acids and other oils by spray drying with an in situ internal gelation mechanism achieving cross-linking of polymers during the process, which is well-suited for industrial scale-up. Spray drying formulations of a mixture of an immiscible hydrophobic cargo and an emulsifier of a hydrophobically modified hydrophilic polymer with a suspension of a multivalent ion cross-linkable polymer, at least one acid, at least one volatile base and at least one salt of a multivalent ion can be adapted to provide control over particle size, degree of crosslinking, enteric release of cargo and shelf life. The methods produce microcapsules that enhance the shelf life of lipophilic bioactives while providing a mechanism of gastrointestinal delivery.

### CROSS-REFERENCE TO RELATED APPLICATIONS

This application claims priority to, and is a 35 U.S.C. § 111(a) continuation of, PCT international application number PCT/US2020/029557 filed on Apr. 23, 2020, incorporated herein by reference in its entirety, which claims priority to, and the benefit of, U.S. provisional patent application Ser. No. 62/837,399 filed on Apr. 23, 2019, incorporated herein by reference in its entirety. Priority is claimed to each of the foregoing applications.

The above-referenced PCT international application was published as PCT International Publication No. WO 2020/219700 A1 on Oct. 29, 2020, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to spray drying encapsulation systems and methods and more particularly to a method for the production and use of cross-linked alginate microcapsules that contain a hydrophobically functionalized starch that enhances the storage stability of the oxygen labile cargo and facilitates controlled release of the cargo in the gastrointestinal tract. Small cross-linked microcapsules are produced in a single step spray drying method, wherein polymer gelation occurs during spray drying upon volatilization of a base and rapid release of otherwise unavailable multivalent ions as the pH is reduced.

### 2. Background

Because the human body does not synthesize substantial quantities of long-chain omega-3 polyunsaturated fatty acids (PUFA), including eicosapentaenoic acid (EPA) and docosahexaenoic acid (DHA), these lipids must be consumed through dietary sources including the consumption of fish or through the use of oil supplements. A variety of health benefits have been associated with the consumption of these lipids including reductions in the risk of type-2 diabetes and cardiovascular disease.

To help meet this dietary need, there is growing interest in the incorporation of PUFA and similar oils into functional foods. However, fortifying foods with fish oil is particularly challenging due to the susceptibility of PUFA and similar fatty acids to oxidation, which induces rancidity that may compromise their bioactivity.

In the food industry, microencapsulated food ingredients are most commonly prepared by spray drying, a highly economical and well-established microencapsulation technology. Food grade wall materials include proteins, oligosaccharides, starches, alginates, and gums. The selection of wall materials may have a profound effect on the oxidative stability of microencapsulated materials.

Microencapsulation is a promising strategy to stabilize fish oil for use as a food ingredient in an attempt to mask unpleasant flavors and odors, protect against oxidation, control the release of cargo, and even enhance bioavailability. However, most spray dried powders are soluble in water and do not provide oxidative protection to the sensitive encapsulated cargo. There is also a need for scalable microencapsulation technologies that can reliably achieve the delayed release of lipophilic bioactives in aqueous environments.

Sodium alginate exhibits unique properties as a microencapsulation wall material. In the presence of calcium ions, alginate forms an electrostatically cross-linked matrix that is insoluble in water. Cross-linked alginate microcapsules exhibit enteric release properties, retaining cargo through the stomach and releasing it in the intestine. Numerous studies have investigated the encapsulation of lipophilic cargo in cross-linked alginate microcapsules at the laboratory scale, and the oxidative stability of lipophilic cargo in these systems.

When incorporated into a functional food, alginate microcapsules may enhance the stability of PUFA during freezing and cooking. Unfortunately, the process of preparing cross-linked alginate microcapsules is challenging to scale industrially. The process would require a series of costly unit procedures, and the alginate gelation step is not well-established at an industrial scale.

Given that lipophilic cargo relevant to the food industry (e.g., PUFA) tends to be susceptible to oxidation, there was an unmet need for providing reliable methods and examining oxidative stability in the context of microencapsulation systems including the crosslinked alginate microcapsule (CLAM) systems.

### BRIEF SUMMARY

Systems and methods are provided for microencapsulating cargos of oxygen sensitive bioactive compounds, such as polyunsaturated fatty acids (PUFA), in dry cross-linked alginate capsules to enhance their shelf life, mask unwanted flavors and odors, facilitate their incorporation into food products, and provide an intestinal release mechanism.

A range of small to large microcapsules can be produced by the methods. Hydrophobically functionalized starch is included in the spray drying formulations to enhance the storage stability of oxygen-labile cargo in spray-dried cross-linked polymer microcapsules as an illustration of the methods. For example, the encapsulation methods and capsules of the present technology provide a substantial increase in the shelf life of microencapsulated omega-3 fatty acids. Relative to conventional processes for preparing alginate/starch microcapsules, the technology provides enhanced scalability.

Conventional processes that are used to prepare microcapsules with similar compositions require a series of unit operations to form, cross-link, and dehydrate the microcapsules. The present technology prepares microcapsules in fewer unit operations, via industrially scalable processes. Relative to other methods that do not utilize cross-linked polymers, this technology yields particles that remain largely insoluble in water as well as in simulated gastric fluid.

The microencapsulation technology of alginate cross-linking formulations is compatible with highly scalable spray-drying unit operations. By implementing pH control over a formulation containing a volatile base and a weak acid, alginate crosslinking occurs via in situ internal gelation during spray drying. The volatile base vaporizes during spray drying, and the pH of the atomized droplets decreases, liberating calcium ions from an acid-soluble salt to cross-link the alginate and entrap cargo. This in situ internal gelation process for producing crosslinked alginate microcapsules (CLAMs) is more economically feasible due to the consolidation of particle formation, gelation, and drying steps into a single unit procedure. The extent of cross-linking in CLAMs can also be modulated via the acid-soluble salt content.

In one embodiment, the encapsulant or film forming composition comprises a mixture of an immiscible hydrophobic cargo, a hydrophobically-modified hydrophilic polymer, a cross-linkable polymer, an acid, at least one volatile base and a salt of a multivalent ion and water. The pH of the mixture is selected so that the salt of the multivalent ion is insoluble and volatilization of the volatile base during spray drying liberates multivalent ions and initiates cross-linking of the polymer molecules to form the capsule or film.

In one embodiment, hydrophobic oxygen-sensitive cargo is emulsified in an aqueous solution containing hydrophobically functionalized starch as a surfactant. The emulsion is then mixed with a cross-linkable (poly-ionic) polymer solution, insoluble cross-linker salt, and an organic acid titrated to neutral pH by the addition of a volatile base. Atomization of the liquid suspension (e.g. spray-drying) produces a fine powder of dry microcapsules where the oxygen-sensitive cargo is protected by a matrix of cross-linked polymer and the presence of a hydrophobized starch. The starch/polymer microcapsules confer enhanced oxidative stability to their cargo relative to cross-linked polymer microcapsules prepared without modified starch. Furthermore, microcapsules prepared with high hydrophobized starch content retained enteric release properties that are intrinsic to the cross-linked polymer system.

While the cross-linked alginate microcapsule (CLAMS) process can contribute controlled release properties, the capsules produced by this process alone are not particularly good at extending the shelf-life of oxygen sensitive cargo. The OSA-starch (or other hydrophobically-modified hydrophilic polymer) is good at extending shelf-life of oxygen sensitive cargo but does not confer controlled release properties to the microcapsules. The combination of the two processes synergistically produces microcapsules that both extends the shelf-stability of oxygen sensitive cargo and controls the conditions of the release of the cargo. This enteric release property is a particularly useful feature of the microcapsules.

Furthermore, it should be emphasized that the hydrophobically-modified starch is not required to be the emulsifier component of the formulation; it can be just an additional ingredient in the formulation which confers oxidative stability to the cargo. However, in one preferred embodiment the selected starch component is the emulsifier the spray dried formulation.

According to one aspect of the technology, a two-step formulation mixing, one-step encapsulation process is provided that is industrially scalable, predictable and has low operating costs.

Another aspect of the technology is an encapsulation system for the food industry that enhances the shelf life of lipophilic bioactives while providing a mechanism of controlled gastrointestinal delivery.

A further aspect of the technology is to provide stable spray drying compositions that are suitable for conventional industrial spray dryers.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a functional block diagram of a method for fabricating microcapsules, fibers or films with a spray drying formulation including a hydrophobically functionalized starch according to one embodiment of the technology.

FIG. 2A is a graph depicting stability of EPA in 25% fish oil OSA-starch/CLAMs during storage under accelerated conditions.

FIG. 2B is a graph depicting stability of EPA in 25% fish oil OSA-starch/CLAMs during storage under ambient conditions.

FIG. 2C is a graph of the EPA retained in (or extractable from) CLAMs after spray-drying is given as a percentage of theoretical EPA content. (Accelerated conditions: 37° C., loosely capped, in the dark. Ambient conditions: Room temperature, head space of vial flushed with Argon and then cap tightly closed, in the dark).

FIG. 3A is a graph depicting shelf stability of omega-3's in non-encapsulated fish oil under accelerated conditions where material was stored in the dark at 37° C. in loosely-capped vials.

FIG. 3B is a graph depicting shelf stability of omega-3's in non-encapsulated fish oil under ambient conditions where material was stored in the dark at 25° C. in tightly-capped vials flushed with an inert gas.

FIG. 4 is a graph depicting the percentage of fish oil cargo released from microcapsules after a 2 h incubation (with agitation) in water, simulated gastric fluid (SGF), and simulated intestinal fluid (SIF). SGF was 0.2% sodium chloride adjusted to pH 1.5 with HCl. SIF was 50 mM phosphate buffer at pH 7.4.

### DETAILED DESCRIPTION

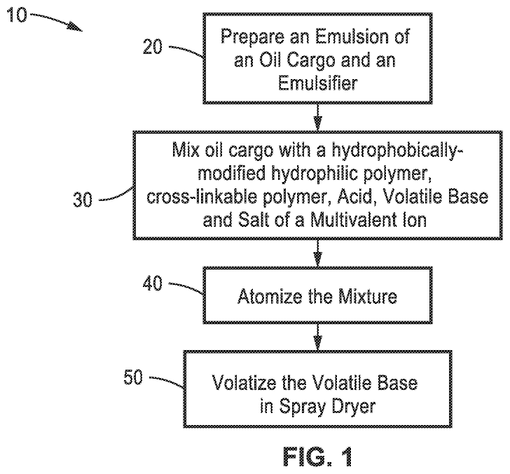

Referring more specifically to the drawings, for illustrative purposes, systems and methods for encapsulating oxygen sensitive cargo are generally shown. Several embodiments of the technology are described generally in FIG. 1 to FIG. 4 to illustrate the characteristics and functionality of the devices, systems and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

An alginate encapsulation of an oxygen sensitive fish oil cargo is used to generally illustrate the methods and resulting microcapsule product characteristics. In this illustration, an oil cargo is emulsified with a hydrophobically functionalized starch, included in the formulation to enhance the storage stability of oxygen-labile cargo in spray-dried cross-linked polymer microcapsules. The prepared emulsion is then mixed with an aqueous formulation that contains sodium alginate, a calcium salt that is only soluble at reduced pH and an organic acid that has been neutralized to a pH just above the pKa with a volatile base. Calcium ions that are needed for cross-linking become available during spray drying by volatilization of the volatile base and the consequent drop in the pH of the spraying solution permitting cross-linking of the alginate polymer in this embodiment. Alginate cross-linking confers controlled release properties to the resulting microparticles and the formation of an alginate acid gel at gastric pH, enables enteric release of the encapsulated cargo. Although the methods are demonstrated in the context of alginate encapsulation, the apparatus and methods can be adapted and applied to the use of other polymer materials as well.

Turning now to FIG. 1, an embodiment of the method 10 for the stable encapsulation of oxygen-labile cargo is shown schematically. At block 20, an emulsion of an oxygen sensitive cargo such as a fish oil mixed with an emulsifier is formed. Suitable emulsifiers include whey protein isolate (WPI), Tween 80, lecithin, casein and a hydrophobically-modified hydrophilic polymer such as n-octenyl succinic anhydride modified starch (OSA-starch). OSA-starch is particularly preferred as an emulsifier. If a different emulsifier is used to emulsify the oil cargo, OSA-starch (or other hydrophobically-modified hydrophilic starch) may also be added to the formulation after the emulsification step.

At block 30, the emulsion is then mixed with a solution for capsule formation of at least one multivalent ion cross-linkable polymer, an acid, a volatile base and a salt of a multivalent ion. A monomer, polymer or other unit that can be cross-linked in the presence of multivalent ions can be used at block 30. The hydrophobically-modified hydrophilic polymer can be added at this step to enhance oxidative stability of the oxygen sensitive cargo.

Suitable cross-linkable polymers in the initial capsule formation solution of block 30 can include organic polymers and proteins such as alginate, chitosan, collagen, polygalacturonates (pectins), carboxymethylcellulose, hyaluronic acid, soy and whey proteins. Chemically modified starches are also cross-linkable polymers, and they may make up a substantial portion of the capsule. The capsule polymer matrix can also be formed from a mixture of such polymers (e.g. alginates and proteins). The polymers selected at block 30 can also be a combination of cross-linkable polymers and co-polymers. Formulations with a mix of cross-linkable polymer types can also be used that may improve protection of the sensitive encapsulated materials.

The term polymer is used in the general sense to refer to the molecular entity or unit that has at least one functional cross-linkable moiety that physically or chemically cross-links in the presence of a multivalent ion and the terms are not intended to be limiting. The polymerized unit may be a high molecular weight polymer or may be oligomeric. Any molecule that cross-links with multivalent ions is a candidate for the polymer selection.

The acid that is selected for the formulation at block 30 is preferably matched with the volatile base that is selected so that cross-linking will occur with the polymers as a result of the volatilization of the base during spray drying or deposition. In one embodiment, an anti-oxidative acid is used instead of or in combination with the organic acid in the formulation to increase protection of oxygen-sensitive cargo.

Suitable acids that are selected for use at block 30 include acetic acid, acrylic acid, adipic acid, ascorbic acid, gallic acid, glutaric acid, succinic acid and caffeic acid. Other suitable acids include malic acid, citrate, and lactic acid. The acid that is selected for the formulation at block 30 of FIG. 1 is preferably an acid with a pKa in the 4 to 5.5 range.

The volatile base that is selected for use at block 30 preferably includes ammonium hydroxide, and other volatile amines such as methylamine, dimethylamine, trimethylamine, ethylamine, diethylamine, isobutylamine, ethylenediamine, N,N-diisopropylethylamine, morpholine, and piperazine.

The source of multivalent cations or crosslinking agents in the capsule solution are preferably acid water-soluble salts that initiate cross-linking. Of the multivalent ions that are capable of cross-linking polymers, divalent ions and trivalent ions are particularly preferred. Any salt of a divalent or trivalent ion that is soluble only under acidic conditions can be selected at block 30 and used in the formulation. For example, salts of barium (Ba²⁺), beryllium (Be²⁺), calcium (Ca²⁺), chromium (Cr²⁺), cobalt (Co²⁺), copper (Cu²⁺), iron (Fe²⁺), lead (Pb²⁺), magnesium (Mg²⁺), mercury (Hg²⁺), strontium (Sr²⁺), tin (Sn²⁺), and zinc (Zn²⁺) can be used. However, dicalcium phosphate, calcium carbonate, calcium oxalate salts are particularly preferred.

The selected acids, bases, salts and cargo emulsion that are mixed together to produce the final formulation at block 30 are then atomized and preferably spray dried at block 40 of FIG. 1. The quantities of each component of the composition are determined by the pH of the resulting formulation and can be optimized. The formulation must have a pH that maintains the selected multivalent salt as an insoluble salt until liberation by the volatilization of the base.

The formulation of emulsion and encapsulant solution mixture is preferably atomized in a spray dryer or spinning disc dryer or other device that will produce droplets of a desired diameter at block 40. In one other embodiment, the components of the final formulation are divided and mixed at the nozzle head at the time of atomization at block 40. Volatilization of the volatile base at block 50 will cause capsule formation from the droplets produced at block 40. The operational parameters of the spray drying apparatus can also be controlled to affect the capsule properties such as particle-size and particle-size distribution.

The volatilization of the volatile base of the droplets during flight at block 50 changes the pH of the formulation allowing the salt to disassociate so that multivalent ions become available for cross-linking of the polymers and capsule formation. Further cross-linking can be achieved at block 50 by increasing the temperature to enhance the volatilization of the volatile base. Enhanced base volatilization leads to a more rapid and complete pH drop, which leads to greater divalent ion dissolution and enhanced cross-linking.

Although the methods and mechanism are illustrated in the context of spray drying, the methods can be adapted to use in other setting such as with a fluidized bed granulator or other spray granulator schemes, and film forming schemes such as fluidized bed spray coaters.

It can be seen that the formulations and methods provide a number of variables and component selections that allow them to be adapted to a variety of encapsulation or coating applications and control over capsule characteristics, shelf life and enteric release properties. For example, the degree of cross-linking and associated release rate of the encapsulated active ingredient cargo can be controlled by the choice of polymer, volatile base and encapsulation conditions. Emulsifier type may also be a factor influencing the stability of the cargo and the release properties of the cargo from the capsules in desired physiological conditions.

The technology described herein may be better understood with reference to the accompanying examples, which are intended for purposes of illustration only and should not be construed as in any sense limiting the scope of the technology described herein as defined in the claims appended hereto.

### Example 1

In order to demonstrate the functionality of the methods, an oxygen-sensitive hydrophobic cargo (fish oil) was emulsified in a solution of hydrophobically functionalized starch (n-octenyl succinic anhydride modified starch (OSA-starch). This emulsion was blended 1:1 (wt/wt) with a suspension of 4% (w/w) sodium alginate (cross-linkable polymer), 1% (w/w) dicalcium phosphate dihydrate (cross-linking agent), and 2% (w/w) succinic acid titrated to pH 5.6 with ammonium hydroxide. The composition of fish oil in the emulsion was selected to achieve 25 g fish oil per 100 g of dry powder. The modified starch content of the emulsion was determined based on the target ratio of modified starch to cross-linkable polymer (sodium alginate). Several embodiments of this invention were produced, at varying ratios of modified starch to cross-linkable polymer (8:2, 4:2, and 2:2 starch/alginate).

In order to prepare the microcapsules with the same dry powder oil content (i.e. 25%), the oil:starch ratio in the emulsion was varied. For example, 8:2 starch:alginate CLAMs were prepared by first homogenizing an emulsion of 7.66% fish oil and 16% starch. This emulsion was subsequently combined 1:1 with the alginate/succinic acid/calcium phosphate suspension as described. Cross-linking of alginate polymer occurred during the spray-drying of the suspensions to form powdered microcapsules. Cross-linking of the polymers occurred during the spray-drying of the suspensions to form powdered microcapsules.

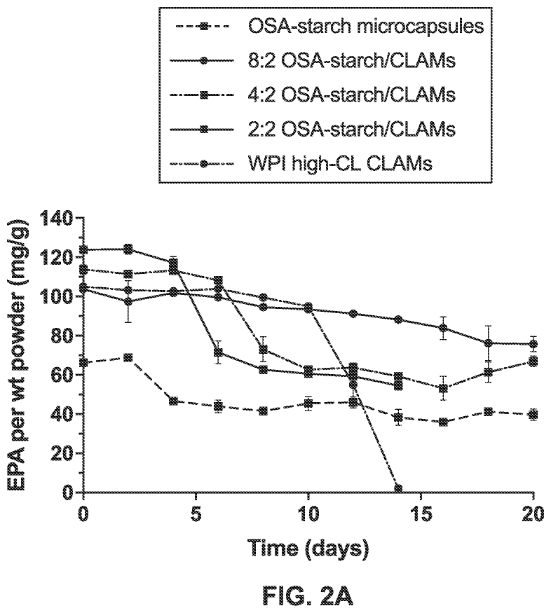

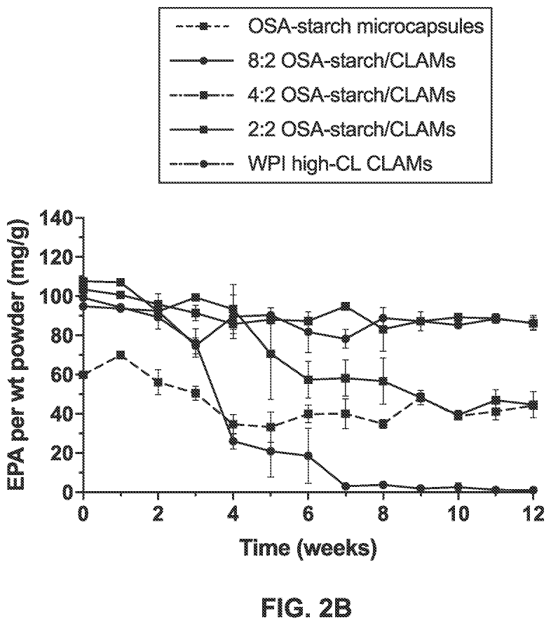

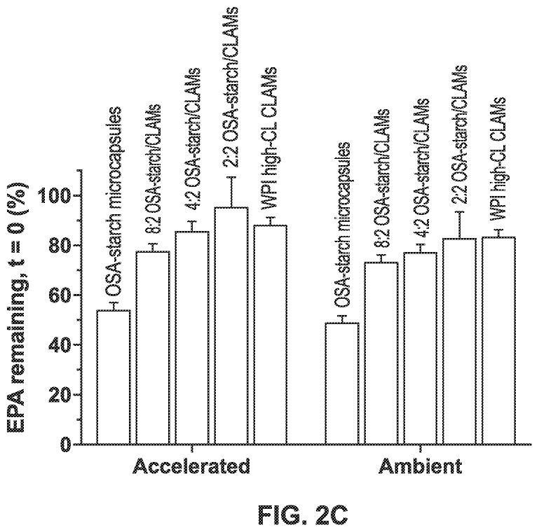

The stability of the omega-3 fatty acids that are intrinsic to fish oil (eicosapentaenoic acid (EPA) and docosahexaenoic acid (DHA)) were monitored during the storage of the powders. The stability of EPA in 25% fish oil OSA-starch/CLAMs during storage under was evaluated and shown for the accelerated conditions (FIG. 2A) and in ambient conditions (FIG. 2B). The EPA retained in (or extractable from) CLAMs after spray-drying is given as a percentage of theoretical EPA content in two separate batches is shown in FIG. 2C.

The tested microcapsules combining OSA-starch and in situ cross-linked alginate exhibited an extended duration of DHA and EPA stability relative to control powders prepared with either OSA-starch or cross-linked alginate as the microencapsulation matrix. EPA levels in OSA-starch/CLAMs remained elevated for a longer time period compared to the control microcapsules (DHA levels were highly correlated to EPA levels). The duration of chemical stability of cargo increased as the ratio of modified starch to alginate increased (FIG. 2A-FIG. 2C).

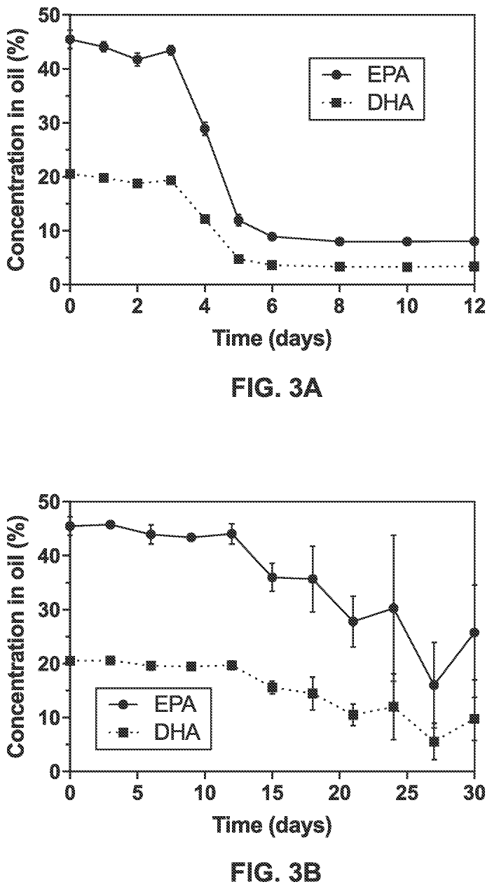

The tested starch/alginate microcapsules also exhibited elevated content of DHA and EPA over the duration of the time of storage, relative to non-encapsulated fish oil stored under the same conditions as shown in FIG. 3A to FIG. 3B.

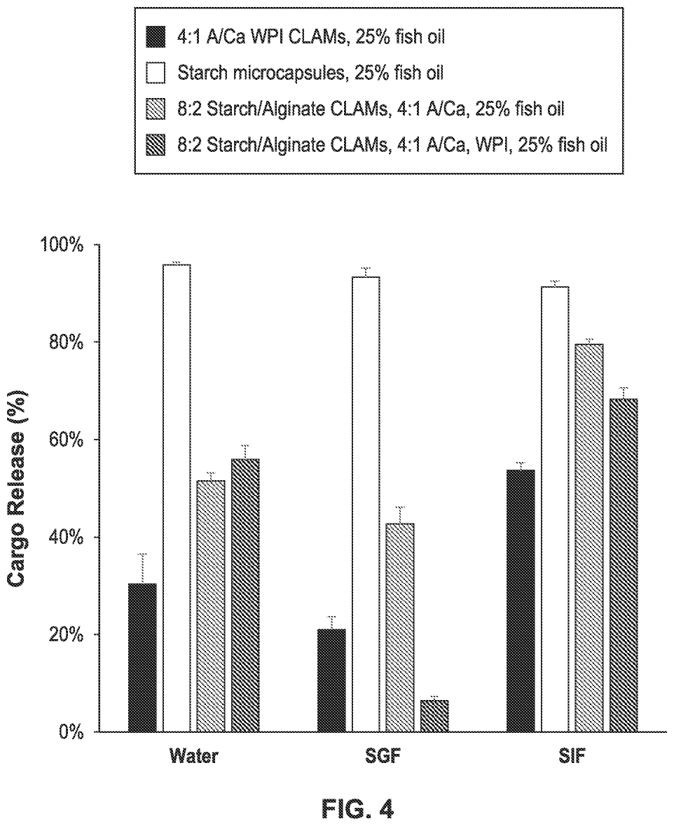

Unlike OSA-starch microcapsules, CLAMs formulated with OSA-starch and alginate exhibited enteric release behavior. As shown in FIG. 4, the OSA-starch microcapsules completely released cargo in water, simulated gastric fluid (SGF), and simulated intestinal fluid (SIF). OSA-starch/CLAMs prepared with 8:2 OSA-starch/alginate released less than half their cargo in SGF but nearly completely in SIF. This enteric release behavior was similar to that observed for control CLAMs prepared without OSA-starch. Despite comprising mostly OSA-starch, the OSA-starch/CLAMs exhibited release behavior more typical of CLAMs than the non-enteric release of OSA-starch microcapsules.

Retention of cargo in SGF was further enhanced by incorporating whey protein into the OSA-starch/CLAMs formulation as illustrated in FIG. 4. In this case, a 6% fish oil emulsion was prepared with 1.2% whey protein isolate, which was homogenized and added to the infeed suspension containing OSA-starch, alginate, succinic acid, ammonium hydroxide, and calcium phosphate.

### Example 2

To further demonstrate the stability of the cargo and capsules formed by encapsulation methods using different emulsifiers for comparison, an emulsion premix containing 1.2% emulsifier (Tween 80 or whey protein isolate-WPI) and 6% fish oil was blended using a rotor-stator at 15,000 rpm for 2 min. This coarse emulsion was subsequently passed 4 times through a high-pressure homogenizer (BEE International, South Easton, Mass.) operated at 25,000 psi, yielding a fine emulsion. This emulsion was added to an aqueous suspension of sodium alginate, succinic acid (titrated to pH 5.6 using ammonium hydroxide), and insoluble CaHPO₄ and subsequently fed into a B-290 laboratory spray dryer (Buchi, New Castle, Del.). All spray-drying inlet suspensions contained 2% sodium alginate, 1% succinic acid, and either 0.5% or 0.1% CaHPO₄ (to achieve either high or low cross-linking. Spray dryer operating conditions were as follows: inlet air temperature was set to 150° C., aspirator airflow rate was set to maximum (35 m³/h), peristaltic pump was set to 20% of maximum (6 mL/min), and nozzle air flow was set to two-thirds of maximum (40 mm on Q-flow indicator). Spray-dried CLAMs were prepared targeting an oil content of 25% (w/w) in the dry powders.

DHA and EPA concentrations in the sample extracts were determined using a model GC-2010 Plus Gas Chromatograph equipped with AOC-20i auto injector (Shimadzu, Kyoto, Japan). Retention of EPA and DHA was calculated to evaluate the loss of PUFA during spray drying. Retention was expressed as a percentage, defined as the powder's PUFA content on day 0 (mg/g) normalized by the expected PUFA content (mg/g).

Cross-linked alginate microcapsules (CLAMs) containing fish oil without emulsifiers were prepared by spray drying for comparison to determine the impact of the emulsifier choice and alginate cross-linking on PUFA stability. The feed emulsions were formulated such that cross-linking of alginates was achieved during the spray-drying process. Upon atomization at the nozzle, the vaporization of ammonia decreased the pH of the dehydrating droplets. Acid-soluble CaHPO₄ dissolved as a result, liberating calcium ions to cross-link the alginate matrix. The extent of cross-linking that occurred in this process was adjusted via the CaHPO₄ content in the feed: high-CL CLAMs were prepared with 0.5% CaHPO₄ in the feed, while low-CL CLAMs were prepared with 0.1% CaHPO₄ in the feed. The choice of emulsifier was varied in conjunction with these two cross-linking levels.

Fish oil in water emulsions with similar submicron particle size distributions were produced with either Tween 80 or WPI. Spray-drying emulsions in this size range has previously been shown to produce powders with desirable attributes, including reduced surface oil content and reduced rate of oxidation.

The chemical stability of EPA and DHA in spray-dried CLAMs was evaluated during storage under two environmental conditions and benchmarked against non-encapsulated fish oil. The initial EPA and DHA concentrations in fish oil were 45.5±1.7% and 20.5±0.7%, respectively. Without encapsulation, EPA and DHA concentrations in fish oil remained unchanged for 3 days under accelerated conditions and 12 days under ambient conditions, after which their concentrations began to decline. Under accelerated conditions, PUFA concentrations dropped precipitously over the span of 2 days, in contrast to the more gradual decline that occurred under the lower temperature, oxygen-limited ambient condition.

### Example 3

For comparison, microcapsules were prepared according to the methods illustrated in FIG. 1 with OSA-starch incorporated into the alginate matrix of CLAMs to bolster their protective properties and enhance the shelf stability of encapsulated fish oil and evaluated. The combination of alginate and starch as a composite wall material was shown to enhance the stability of microencapsulated lipophilic cargo. The dense packing of the OSA-starch in the matrix may reduce microcapsule porosity, oxygen diffusivity, and thus the rate of cargo oxidation.

Capsules were prepared with varying OSA-starch to alginate ratios, targeting 25% fish oil in dry powder. With the exception of the OSA-starch microcapsule control powder, all powders were prepared according to high-crosslinking specifications. For these powders, the OSA-starch also functioned as the emulsifier and no additional emulsifier was included in the OSA-starch CLAM formulations. Fish oil emulsions stabilized by OSA-starch exhibited submicron particle sizes distributions prior to alginate addition and subsequent spray drying.

The inclusion of OSA-starch into 25% fish oil CLAM formulations was motivated by the goal of increasing the shelf life of the fish oil powders. Incorporation of increasing quantities of OSA-starch lengthened the duration of PUFA stability during storage under accelerated and ambient conditions.

Under accelerated conditions, while the 2:2 and 4:2 OSA starch/CLAM microcapsules were stable for 4 and 6 days (respectively) before the onset of EPA and DHA depletion, the EPA and DHA contents of 8:2 OSA-starch/CLAMs remained elevated over 14 days.

When stored under ambient conditions, the EPA and DHA contents of both 8:2 and 4:2 OSA-starch/CLAMs remained elevated over the duration of the 12-week study, but the 2:2 OSA-starch/CLAMs began to experience depletion after 4 weeks. Thus, both the accelerated and ambient storage stability data suggest that increasing the OSA-starch content of CLAMs lengthened the duration of PUFA stability.

Composite OSA-starch/alginate microcapsules appear to provide enhanced protection against lipid oxidation relative to microcapsules composed of OSA-starch alone. The improvement of oxidative stability with increasing OSA-starch content can be linked to the differences in powder physicochemical properties. As the OSA-starch content of microcapsules (and total solids in the liquid formulation) increased, their surface oil content was observed to decrease. SEM images of OSA-starch/CLAMs supported the surface oil measurements. OSA-starch microcapsules featured surfaces that essentially lacked submicron dimples left by vaporizing surface oil droplets. As the OSA-starch content in the formulations was decreased, the surfaces of OSA-starch/CLAMs exhibited a greater prevalence of submicron-sized dimples, indicating greater surface oil. The reduction of surface oil with increasing OSA-starch content may be attributed to the increase in total solids, which may slow the migration of lipid droplets to the surface during spray drying, resulting in less surface oil and enhanced cargo stability.

In sum, increasing the OSA-starch content in OSA-starch/CLAMs increased the duration of PUFA stability. Under ambient conditions, 8:2 and 4:2 OSA-starch/CLAMs exhibited extended PUFA stability over 3 months of storage, which constitutes a considerable improvement over the previous formulations.

### Example 4

Designing microencapsulation systems to target the delivery of bioactive cargo to specific sites in the gastrointestinal tract is a potential approach to enhance bioavailability when designing functional foods. Enteric release systems retain cargo in the acidic conditions of the stomach and release it in the intestinal environment, which is the site of absorption for many nutrients and bioactives. This enteric release property, which is a key feature of alginate-based microcapsule formulations, provides the benefit of protecting the cargo from degradation that may occur in the stomach and releasing the cargo at the site of absorption.

To demonstrate control over the parameters that influence release of lipid cargos from microcapsules, high and low percentage cross-linked microcapsules were prepared and the enteric release of cargo in simulated gastric fluid (SGF) and 90% in simulated intestinal fluid (SIF) was evaluated.

Previous work with low-CL microcapsules with Tween 80 emulsifier exhibited enteric release of corn oil cargo, releasing approximately 20% in simulated gastric fluid (SGF) and 90% in simulated intestinal fluid (SIF). By comparison, the low-CL microcapsules prepared with WPI released slightly less oil cargo in SGF and SIF, likely due to interactions between protein emulsifier and the alginate matrix.

Increasing the extent of alginate cross-linking in the CLAM microcapsules lessened the release of oil in water and SIF, likely due to the formation of a less permeable matrix. However, high-CL microcapsules released more oil in SGF relative to the low-CL CLAMs. It is believed that at low pH, insoluble calcium particles dissolve, leaving pores that facilitate cargo release from alginate matrices.

While CLAMs exhibited enteric release of fish oil cargo, microcapsules prepared entirely of OSA-starch dissolved and released over 90% of fish oil cargo in water, SGF, and SIF. On the other hand, 8:2 OSA-starch/CLAMs released just over 50% of fish oil cargo into water, just over 40% in SGF, and just under 80% in SIF. Despite being composed predominantly of OSA-starch, the 8:2 OSA-starch/CLAMs exhibited enteric release properties, retaining more than half of their cargo under gastric conditions but releasing most of their cargo under intestinal conditions. Despite being a minor constituent in the composition, the cross-linked alginate mitigated the release of fish oil cargo in this system. The in vitro cargo release properties demonstrated here suggest that CLAMs formulated with OSA-starch is a suitable microcapsule system for targeting the delivery of cargo to the site of nutrient absorption in the gastrointestinal tract.

Furthermore, unlike microcapsules prepared with OSA-starch alone but similar to CLAMs, OSA-starch/CLAMs exhibited enteric release properties, retaining cargo under simulated gastric conditions and releasing it under intestinal conditions. Overall, encapsulation in CLAMs formulated with OSA-starch appears to be an important approach for the food industry to enhance the shelf life of lipophilic bioactives while providing a mechanism of gastrointestinal delivery.

From the description herein, it will be appreciated that the present disclosure encompasses multiple embodiments which include, but are not limited to, the following:

1. An encapsulant or film forming composition, comprising: (a) a mixture of an immiscible hydrophobic cargo, a hydrophobically-modified hydrophilic polymer, a multivalent ion cross-linkable polymer, an acid, at least one volatile base, a salt of a multivalent ion and water; (b) wherein the pH of the mixture is poised such that the said salt of a multivalent ion is insoluble; (c) wherein volatilization of said volatile base liberates multivalent ions and initiates cross-linking of the polymer molecules.

2. The composition of any preceding or following embodiment, wherein the hydrophobically modified hydrophilic polymer comprises n-octenyl succinic anhydride (OSA) modified starch.

3. The composition of any preceding or following embodiment, wherein said multivalent ion cross-linkable polymer is a polymer selected from the group consisting of alginate, chitosan, collagen, polygalacturonates (pectins), hyaluronic acid, carboxymethylcellulose, soy and whey proteins.

4. The composition of any preceding or following embodiment, wherein said acid is an organic acid selected from the group of acids consisting of adipic acid, acrylic acid, glutaric acid, citrate, succinic acid, ascorbic acid, gallic acid, malic acid, lactic acid, acetic acid and caffeic acid.

5. The composition of any preceding or following embodiment, wherein said volatile base is ammonium hydroxide.

6. The composition of any preceding or following embodiment, wherein said volatile base is selected from the group of volatile amine bases consisting of methylamine, dimethylamine, trimethylamine, ethylamine, diethylamine, isobutylamine and ethylenediamine.

7. The composition of any preceding or following embodiment, wherein said multivalent ion is a divalent cation.

8. The composition of any preceding or following embodiment, wherein said divalent cation is selected from the group of cations consisting of barium (Ba2+), calcium (Ca2+), chromium (Cr2+), copper (Cu2+), iron (Fe2+), magnesium (Mg2+) and zinc (Zn2+).

9. A method of forming a capsule of multivalent ion cross-linkable polymer to enhance storage stability of encapsulated oxygen-labile cargo, the method comprising: (a) forming a dispersion of an oxygen-labile cargo and a hydrophobically-modified hydrophilic polymer; (b) mixing said dispersion with a suspension comprising multivalent ion cross-linkable polymer, at least one salt of an acid-soluble multivalent ion and an acid neutralized with a volatile base with the emulsion; and (c) volatilizing said volatile base of said mixture, thereby liberating said multivalent ions and initiating cross-linking of the cross-linkable polymer molecules.

10. The method of any preceding or following embodiment, wherein the hydrophobically modified hydrophilic polymer comprises n-octenyl succinic anhydride (OSA) modified starch.

11. The method of any preceding or following embodiment, wherein said multivalent ion cross-linkable polymer is selected from the group of polymers consisting of alginates, polygalacturonates, chitosan, collagen, hyaluronic acid, carboxymethylcellulose, soy proteins and whey proteins.

12. The method of any preceding or following embodiment, wherein said multivalent ion is a divalent cation is selected from the group of cations consisting of barium (Ba2+), calcium (Ca2+), chromium (Cr2+), copper (Cu2+), iron (Fe2+), magnesium (Mg2+) and zinc (Zn2+).

13. The method of any preceding or following embodiment, wherein said acid is an organic acid selected from the group of acids consisting of adipic acid, acrylic acid, glutaric acid, succinic acid, ascorbic acid, gallic acid, malic acid, lactic acid, acetic acid and caffeic acid.

14. The method of any preceding or following embodiment, wherein said volatile base is selected from the group of volatile amine bases consisting of methylamine, trimethylamine, ethylamine, diethylamine and triethylamine, isobutylamine, N,N-diisopropylethylamine, morpholine, piperazine, and ethylenediamine.

15. An encapsulant or film forming composition, comprising: (a) a mixture of an immiscible hydrophobic cargo and an emulsifier of a hydrophobically modified hydrophilic polymer with a suspension of a multivalent ion cross-linkable polymer, at least one acid, at least one volatile base and at least one salt of a multivalent ion; (b) wherein volatilization of said at volatile base liberates multivalent ions and initiates cross-linking of the polymer molecules.

16. The composition of any preceding or following embodiment, wherein the hydrophobically modified hydrophilic polymer comprises n-octenyl succinic anhydride (OSA) modified starch.

17. A method of forming a capsule of cross-linked polymer molecules to enhance storage stability of encapsulated oxygen-labile cargo, the method comprising: (a) forming a dispersion of an oxygen-labile cargo and an hydrophobically modified hydrophilic polymer; (b) mixing a multivalent ion cross-linkable polymer, at least one salt of an acid-soluble multivalent ion and an acid neutralized with a volatile base with the emulsion; and (c) volatilizing said volatile base of said mixture, thereby liberating said multivalent ions and initiating cross-linking of the monomer molecules.

18. The method of any preceding or following embodiment, wherein said multivalent ion cross-linkable polymer is selected from the group consisting of alginates, polygalacturonates, chitosan, collagen, carboxymethylcellulose, soy proteins and whey proteins.

19. The method of any preceding or following embodiment, wherein the hydrophobically modified hydrophilic polymer comprises n-octenyl succinic anhydride (OSA) modified starch.

20. The method of any preceding or following embodiment, wherein said volatile base is ammonium hydroxide.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An encapsulant or film forming composition, comprising:
(a) a mixture of an immiscible hydrophobic cargo, a hydrophobically-modified hydrophilic polymer, a multivalent ion cross-linkable polymer, an acid, at least one volatile base, a salt of a multivalent ion and water;
(b) wherein the pH of the mixture is poised such that the said salt of a multivalent ion is insoluble;
(c) wherein volatilization of said volatile base liberates multivalent ions and initiates cross-linking of the polymer molecules.

2. The composition of claim 1, wherein the hydrophobically modified hydrophilic polymer comprises n-octenyl succinic anhydride (OSA) modified starch.

3. The composition of claim 1, wherein said multivalent ion cross-linkable polymer is a polymer selected from the group consisting of alginate, chitosan, collagen, polygalacturonates (pectins), hyaluronic acid, carboxymethylcellulose, soy and whey proteins.

4. The composition of claim 1, wherein said acid is an organic acid selected from the group of acids consisting of adipic acid, acrylic acid, glutaric acid, citrate, succinic acid, ascorbic acid, gallic acid, malic acid, lactic acid, acetic acid and caffeic acid.

5. The composition of claim 1, wherein said volatile base is ammonium hydroxide.

6. The composition of claim 1, wherein said volatile base is selected from the group of volatile amine bases consisting of methylamine, dimethylamine, trimethylamine, ethylamine, diethylamine, isobutylamine and ethylenediamine.

7. The composition of claim 1, wherein said multivalent ion is a divalent cation.

8. The composition of claim 7, wherein said divalent cation is selected from the group of cations consisting of barium (Ba²⁺), calcium (Ca²⁺), chromium (Cr²⁺), copper (Cu²⁺), iron (Fe²⁺), magnesium (Mg²⁺) and zinc (Zn²⁺).

9. A method of forming a capsule of multivalent ion cross-linkable polymer to enhance storage stability of encapsulated oxygen-labile cargo, the method comprising:
(a) forming a dispersion of an oxygen-labile cargo and a hydrophobically-modified hydrophilic polymer;
(b) mixing said dispersion with a suspension comprising multivalent ion cross-linkable polymer, at least one salt of an acid-soluble multivalent ion and an acid neutralized with a volatile base with the emulsion; and
(c) volatilizing said volatile base of said mixture, thereby liberating said multivalent ions and initiating cross-linking of the cross-linkable polymer molecules.

10. The method of claim 9, wherein the hydrophobically modified hydrophilic polymer comprises n-octenyl succinic anhydride (OSA) modified starch.

11. The method of claim 9, wherein said multivalent ion cross-linkable polymer is selected from the group of polymers consisting of alginates, polygalacturonates, chitosan, collagen, hyaluronic acid, carboxymethylcellulose, soy proteins and whey proteins.

12. The method of claim 9, wherein said multivalent ion is a divalent cation is selected from the group of cations consisting of barium (Ba²⁺), calcium (Ca²⁺), chromium (Cr²⁺), copper (Cu²⁺), iron (Fe²⁺), magnesium (Mg²⁺) and zinc (Zn²⁺).

13. The method of claim 9, wherein said acid is an organic acid selected from the group of acids consisting of adipic acid, acrylic acid, glutaric acid, succinic acid, ascorbic acid, gallic acid, malic acid, lactic acid, acetic acid and caffeic acid.

14. The method of claim 9, wherein said volatile base is selected from the group of volatile amine bases consisting of methylamine, trimethylamine, ethylamine, diethylamine and triethylamine, isobutylamine, N,N-diisopropylethylam ine, morpholine, piperazine, and ethylenediamine.

15. An encapsulant or film forming composition, comprising:
(a) a mixture of an immiscible hydrophobic cargo and an emulsifier of a hydrophobically modified hydrophilic polymer with a suspension of a multivalent ion cross-linkable polymer, at least one acid, at least one volatile base and at least one salt of a multivalent ion;
(b) wherein volatilization of said at volatile base liberates multivalent ions and initiates cross-linking of the polymer molecules.

16. The composition of claim 15, wherein the hydrophobically modified hydrophilic polymer comprises n-octenyl succinic anhydride (OSA) modified starch.

17. A method of forming a capsule of cross-linked polymer molecules to enhance storage stability of encapsulated oxygen-labile cargo, the method comprising:
(a) forming a dispersion of an oxygen-labile cargo and an hydrophobically modified hydrophilic polymer;
(b) mixing a multivalent ion cross-linkable polymer, at least one salt of an acid-soluble multivalent ion and an acid neutralized with a volatile base with the emulsion; and
(c) volatilizing said volatile base of said mixture, thereby liberating said multivalent ions and initiating cross-linking of the monomer molecules.

18. The method of claim 17, wherein said multivalent ion cross-linkable polymer is selected from the group consisting of alginates, polygalacturonates, chitosan, collagen, carboxymethylcellulose, soy proteins and whey proteins.

19. The method of claim 17, wherein the hydrophobically modified hydrophilic polymer comprises n-octenyl succinic anhydride (OSA) modified starch.

20. The method of claim 17, wherein said volatile base is ammonium hydroxide.
